(19) United States
(12) Patent Application Publication
(10) Pub. No.: US 2024/0228927 A1
(43) Pub. Date: Jul. 11, 2024

(54) MICROFLUIDIC PLATFORM FOR DETECTION OF LIVER INJURY

(71) Applicant: Javelin Biotech, Inc., Woburn, MA (US)

(72) Inventor: Murat Cirit, Cambridge, MA (US)

(21) Appl. No.: 18/405,741

(22) Filed: Jan. 5, 2024

Related U.S. Application Data
(60) Provisional application No. 63/478,602, filed on Jan. 5, 2023.

(51) Int. Cl. C12M 3/06; C12M 1/00; C12M 1/34; G01N 33/50

(52) U.S. Cl. CPC C12M 23/16; C12M 23/22; C12M 29/14; C12M 29/24; C12M 41/32; C12M 41/34; G01N 33/5067; G01N 2333/765; G01N 2333/9108; G01N 2333/91188; G01N 2333/916

(57) ABSTRACT

A microfluidic chip is configured to determine DILI parameters. The microfluidic chip includes a cell chamber hosting a tissue culture comprising liver tissue; a reoxygenation chamber configured to add oxygen to a fluid media; a fluid loop configured to recirculate the fluid media through the cell chamber and the reoxygenation chamber and supply oxygenated fluid media to the tissue culture; and an oxygen sensor configured to measure an oxygen concentration within the fluid media. The microfluidic chip includes a controller configured to perform operations including recirculating the fluid media in the fluid loop, the fluid media comprising a drug dose; obtaining measurements of the oxygen concentration at a sequence of time points during recirculating; and determining, based on the measured oxygen concentration, at least one physiologic parameter value of the tissue culture that describes a clinical test metric describing a damage level to the tissue culture.

### CLAIM OF PRIORITY

This application claims priority under 35 U.S.C. § 119(e) to provisional U.S. Patent Application Ser. No. 63/478,602, filed on Jan. 5, 2023, the entire contents of which are hereby incorporated by reference.

### TECHNICAL FIELD

This disclosure generally relates to microfluidic devices. More specifically, this disclosure relates to target and metabolomics-based biomarker discovery platforms for diagnosis of drug-induced liver injury (DILI).

### BACKGROUND

DILI is a common cause of acute liver failure and need for transplant, with an estimated incidence of 2.7 cases per 100,000 adults. DILI is the cause of more than half of cases of acute liver failure and a leading cause of failures in clinical development and post-market withdrawals. Analysis and testing of new drugs for potential DILI can be an important step during drug optimization and prior to in-human clinical trials.

The etiology of DILI is complex. DILI can be classified as hepatocellular, causing elevated alanine transaminase (ALT) and aspartate transaminase (AST) levels. DILI can be cholestatic, causing increased alkaline phosphatase (ALP). DILI can be defined as intrinsic (dose-dependent) or idiosyncratic (immune-related). Idiosyncratic DILI is rare, accounting for fewer than 1 case in 10,000. For most cases, generating dose-tox profiles by using escalating doses over time is useful for predicting DILI.

DILI is frequently caused by drug metabolism. There are several mechanisms in which parent drugs or metabolites can cause DILI. A first mechanism includes inhibition of the bile salt export pump (BSEP), which leads to cholestatic injury via reduced bile acid secretion. A second mechanism includes mitochondrial disruption, which occurs when parent drugs and/or their metabolites impact the function of mitochondria and specifically ATP generation and oxygen consumption rates (OCR). A third mechanism includes when reactive (toxic) metabolites react with endogenous proteins to form toxic macromolecular adducts.

### SUMMARY

This disclosure describes microfluidic physiological system (MPS), also called a microfluidic chip or device, configured for performing drug assays on tissue to enable generation of estimates for liver-specific DILI parameters. The MPS includes a cell chamber configured to host liver tissue (e.g., human liver tissue) and emulate an in-vivo assay to enable a data processing system in communication with the MPS to estimate the liver-specific DILI parameters for different drugs. The microfluidic chip includes a cell chamber hosting a tissue culture comprising liver tissue. A reoxygenation chamber configured to add oxygen to a fluid media. A fluid loop configured to recirculate the fluid media through the cell chamber and the reoxygenation chamber and supply oxygenated fluid media to the tissue culture; and an oxygen sensor configured to measure an oxygen concentration within the fluid media. A controller on or in communication with the microfluidic chip is configured to control the pump to recirculate the fluid media in the fluid loop. The fluid media includes a drug dose. The controller obtains measurements of the oxygen concentration at a sequence of time points during recirculating. The controller is configured to determine, based on the measured oxygen concentration, at least one physiologic parameter value of the tissue culture that describes a clinical test metric describing a damage level to the tissue culture.

The microfluidic chips are configured to extend liver tissue culture to at least 28 days, and the cells mature and reach a physiologically relevant state in 5-7 days. The milli-fluidic design of the microfluidic chips described herein provides relatively larger tissue sizes (˜200-250K cells) and media volumes (˜2 milliliters) for multi-scale (e.g., intracellular and extracellular) pharmacological characterization on a single chip (rather than a system of many chips), which enables collection of samples at multiple time points to assess tissue-specific metrics. The microfluidic chip includes a cyclic olefin copolymer (COC) chip material eliminates non-specific binding of lipophilic drugs, in contrast to polydimethylsiloxane (PDMS) chips, and minimizes evaporation of fluid media.

The microfluidic chips and associated systems and processes described herein enable one or more of the following advantages. In typical approaches for assessing DILI, animal models can be substituted for human testing. Animal models can be insufficient for estimation of liver-specific DILI parameters because animal models can fail to accurately reproduce human liver and hepatocellular functions, particularly regarding metabolism and pathophysiology. Animal studies create a large financial burden during lead optimization. To test using animal model, the synthesis of each compound needs to be scaled up from microgram to milligram (μg to mg) quantities before animal studies can be initiated. The scaling-up process can take several months and incur substantial cost without accounting for a cost of the actual animal studies. A prediction of human outcomes using animal models to predict liver toxicity is poor. For example, the animal models have been only 50%, 33%, and 27% successful, reported in monkeys, rats, and dogs, respectively. The microfluidic chips described herein can provide more predictive, cheaper, and faster drug assay outcomes. The microfluidic chips and associated systems and processes described herein can result in more drug candidates succeeding when entering clinical trials by avoiding safety issues.

Additionally, current technologies have limited capacity to predict DILI accurately. Generally, in-vitro hepatic cultures (suspension or plated hepatocytes) are not suitable for comprehensive testing due to short-lived cultures, lasting fewer than 5 days. Generally, suspension cultures die in about four hours. Furthermore, current approaches have a low sensitivity. For example, a sensitivity observed is only about 50% with two-dimensional (2D) hepatic cultures, about 65% with micropatterned hepatic co-culture systems, and between about 48%-69% with the three-dimensional (3D) spheroid cultures. In spheroid cultures, hepatocytes are cultured under chemically defined conditions to form self-aggregating 3D structures. Using human cells, this system can mitigate the species-specific problems seen with animal models. However, a given 3D spheroid assay can detect false negative results at a high percentage (e.g., greater than 30%). Of these false-negatives, a majority can be related to low-clearance drugs that require longer in vitro incubation times to assess their metabolite-based toxicities. The microfluidic chips described herein can enable testing of these low clearance drugs that may otherwise be difficult to assess. For example, in contrast to 3D spheroid cultures, the microfluidic chips described herein can extend the culture period more than 28 days while being suitable for long-term drug incubation and without requiring frequent (e.g., daily) media changes that are needed for 3D spheroid cultures. The microfluidic chips described enable a longer drug incubation time for generating metabolites including reactive (toxic) metabolites. The microfluidic chips described can avoid regions of oxygen-depleted tissue, in contrast to spheroids, which are not ideal for metabolism studies due to their oxygen-depleted cores.

The microfluidic chips described herein are configured to culture functional liver tissue and generate multiscale data for comprehensive DILI assessment. The microfluidic chips described herein provide a stable, reliable in vitro hepatic model system with long-term metabolically functional tissue with physiologically relevant tissue function (albumin, urea) that can generate multi-scale mechanistic information (media & tissue-level, with mechanism-based biomarkers, e.g., mitotoxicity) to assess common DILI phenotypes.

The one or more advantages described can be enabled by one or more aspects or embodiments of the platform. In an aspect, a process for testing cell cultures for drug-induced liver injury (DILI) can include the following. The process includes obtaining a microfluidic chip including a cell chamber hosting a tissue culture comprising liver tissue, a reoxygenation chamber configured to add oxygen to a fluid media, a fluid loop configured to recirculate the fluid media through the cell chamber and the reoxygenation chamber and supply oxygenated fluid media to the tissue culture, and at least one oxygen sensor configured to measure an oxygen concentration within the fluid media. The process includes applying a drug dose to the fluid media of the fluid loop of the microfluidic chip. The process includes incubating the tissue culture in the fluid loop for an incubation period while recirculating the fluid media, comprising the drug dose, in the fluid loop. The process includes measuring the oxygen concentration at a sequence of time points during and after the incubation period. The process includes determining, based on the measured oxygen concentration, at least one physiologic parameter value of the tissue culture that describes a clinical test metric describing a damage level to the tissue culture.

In some implementations, the clinical test metric includes one or more of alanine transaminase (ALT), aspartate transaminase (AST), alkaline phosphatase (ALP), gamma-glutamyl transferase (GGT), serum bilirubin, prothrombin time (PT), an international normalized ratio (INR), a total protein, and an albumin.

In some implementations, the at least one physiologic parameter value of the tissue culture represents a cell viability metric value for the tissue culture.

In some implementations, a media volume in the microfluidic chip includes at least 2 milliliters (ml).

In some implementations, the microfluidic chip is configured for intracellular and extracellular pharmacological characterization on a single microfluidic chip.

In some implementations, the fluid loop comprises a cyclic olefin copolymer (COC) that is configured to reduce or eliminate a non-specific binding of lipophilic drugs.

In some implementations, the microfluidic chip is translucent or transparent, and wherein the microfluidic chip does not cause auto-fluorescence responsive to fluorescence imaging in the cell chamber.

In some implementations, the cell chamber includes a tissue surface area of at least 1 square centimeter (cm2).

In some implementations, the at least one oxygen sensor comprises a first oxygen sensor configured to measure the oxygen concentration in the fluid media at an inlet of the cell chamber, and wherein the at least one oxygen sensor comprises a second oxygen sensor configured to measure the oxygen concentration in the fluid media at an outlet of the cell chamber.

In some implementations, the process includes maintaining the tissue culture for at least 21 days.

In some implementations, the tissue culture comprises at least 200,000 cells.

In some implementations, the process includes determining a mitochondrial function based on the measured oxygen concentration of the tissue culture.

In some implementations, the drug dose comprises one of Sitaxsentan, Clozapine, Diclofenac, Zileuton, Fialuridine, Tolcapone, Asunaprevir, Troglitazone, Telithromycin, Trovafloxacin, Pemoline, Mipomersen, or Nefazodone.

In some implementations, the process includes applying a series of drug doses to the fluid media of the fluid loop of the microfluidic chip during and after the incubation period, the series of drug doses increasing in value.

In some implementations, the process includes applying a series of drug doses to the fluid media of the fluid loop of the microfluidic chip every four days.

In some implementations, the process includes obtaining a plurality of instances of the microfluidic chip, applying drug doses to each microfluidic chip instance of the plurality, a type of the drug dose being different for each microfluidic chip instance, controlling the plurality of instances of the microfluidic chip in parallel.

In an aspect, a microfluidic chip includes a cell chamber hosting a tissue culture comprising liver tissue. The microfluidic chip includes a reoxygenation chamber configured to add oxygen to a fluid media. The microfluidic chip includes a fluid loop configured to recirculate the fluid media through the cell chamber and the reoxygenation chamber and supply oxygenated fluid media to the tissue culture. The microfluidic chip includes at least one oxygen sensor configured to measure an oxygen concentration within the fluid media. The microfluidic chip includes a controller configured to perform operations comprising the following. The operations include recirculating the fluid media in the fluid loop, the fluid media comprising a drug dose. The operations include obtaining measurements of the oxygen concentration at a sequence of time points during recirculating. The operations include determining, based on the measured oxygen concentration, at least one physiologic parameter value of the tissue culture that describes a clinical test metric describing a damage level to the tissue culture.

In some implementations, the clinical test metric includes one or more of alanine transaminase (ALT), aspartate transaminase (AST), alkaline phosphatase (ALP), gamma-glutamyl transferase (GGT), serum bilirubin, prothrombin time (PT), an international normalized ratio (INR), a total protein, and an albumin.

In some implementations, the fluid loop comprises a cyclic olefin copolymer (COC) that is configured to reduce or eliminate a non-specific binding of lipophilic drugs.

In some implementations, the at least one oxygen sensor comprises a first oxygen sensor configured to measure the oxygen concentration in the fluid media at an inlet of the cell chamber, and wherein the at least one oxygen sensor comprises a second oxygen sensor configured to measure the oxygen concentration in the fluid media at an outlet of the cell chamber.

The details of one or more embodiments of these systems and methods are set forth in the accompanying drawings and the description to be presented. Other features, objects, and advantages of these systems and methods are apparent from the description and drawings, and from the claims.

### BRIEF DESCRIPTION OF THE DRAWINGS

FIG. 1 shows a top-view of an example microfluidic chip for performing drug assays on tissue to enable generation of estimates for liver-specific DILI parameters.

FIG. 2 shows a perspective view of an example microfluidic chip for performing drug assays on tissue to enable generation of estimates for liver-specific DILI parameters.

FIG. 3 shows a perspective view of an example platform configured to host a plurality of microfluidic chips shown in FIGS. 1-2 for performing drug assays on tissue to enable generation of estimates for liver-specific DILI parameters.

FIGS. 4A-4C show incubation of liver cells in the microfluidic chip.

FIG. 5 shows a graph of example oxygen levels with the microfluidic chip of FIGS. 1-2.

FIG. 6 shows graphs representing examples predicted and observed hepatic clearances.

FIG. 7 is a flow diagram illustrating example process for performing drug assays on tissue to enable generation of estimates for liver-specific DILI parameters.

FIG. 8 is a block diagram of an example computer system used to provide computational functionalities associated with described algorithms, methods, functions, processes, flows, and procedures described in the present disclosure.

### DETAILED DESCRIPTION

Example microfluidic chips configured for performing drug assays on tissue to enable generation of estimates for liver-specific DILI parameters. The microfluidic chips described herein are configured for recirculation of a fluid media and reoxygenation of cell cultures. This disclosure describes hardware support systems for controlling recirculation of the fluid media and reoxygenation of the cell cultures within the microfluidic chip. The microfluidic chips described herein can be an organ construct house in a microfluidic chip or an organ-on-a-chip (OOC). In some implementations, the cell chambers of the microfluidic chip are called compartments. In the microfluidic chips described herein, the microfluidic chip generally includes a fluid circulation loop for recirculating fluid through a cell chamber including cell tissue of a given cell type or types. Generally, the fluid is continuously recirculated through the cell chamber in a closed loop. The fluid loop generally includes a reoxygenation chamber to reoxygenate oxygen-depleted fluid media recirculating through the fluid loop, as the oxygen is absorbed by the cells of cell chamber. The microfluidic chip design can be based on the oxygen requirements of the cells in the cell chamber and the maximum shear stress tolerances and oxygen absorption rates of those cells, which can limit flow rates of the fluid media, as described throughout the disclosure.

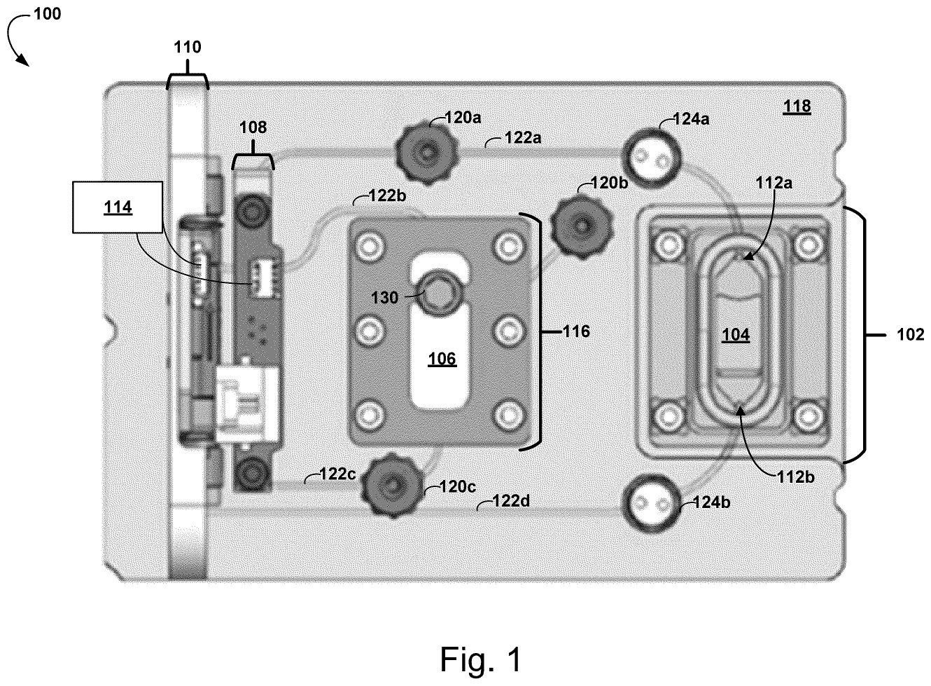

FIG. 1 illustrates a top view of an example microfluidic chip 100 for a recirculating liver chip configured for generation of estimates for liver-specific DILI parameters. The microfluidic chip 100 is configured to emulate organ functionality. The microfluidic chip 100 is configured to enable preclinical drug discovery and assist in developing treatments for various diseases. The microfluidic chip 100 enables long-term tissue mono- or co-culture (e.g., up to at least 28 days).

The microfluidic chip 100 includes a fluid loop configured to circulate fluid media through a cell chamber 104 as subsequently described. The cell chamber 104 is configured to host liver tissue (e.g., human liver tissue) and emulate an in-vivo assay to enable a data processing system in communication with the microfluidic chip to estimate the liver-specific DILI parameters for different drugs. The fluid loop includes a reoxygenation chamber 106 configured to add oxygen to the fluid media. A pump 108 circulates the fluid media through the fluid loop. A flow sensor 110 can measure the flow rate of the fluid media through the fluid loop. The pump 108 is configured to recirculate the fluid media through the cell chamber and the reoxygenation chamber and supply oxygenated fluid media to the tissue culture. An inlet oxygen sensor 112a and an outlet oxygen sensor 112b are each configured to measure an oxygen concentration within the fluid media on either side of the cell chamber 104. A controller 114 on or in communication with the microfluidic chip 100 is configured to control the pump 108 to recirculate the fluid media in the fluid loop and measure the flow rate by the flow sensor 110. In some implementations, the pump is a diaphragm pump 108. In some implementations, the pump 108 is a peristaltic pump.

The fluid media includes a drug dose. The controller 114 obtains measurements of the oxygen concentration from sensors oxygen 112a-b at a sequence of time points during recirculating. The controller 114 is configured to determine, based on the measured oxygen concentration, at least one physiologic parameter value of the tissue culture that describes a clinical test metric describing a damage level to the tissue culture, as described herein.

The microfluidic chip 100 includes a substrate 118 in which various components, such as the pump 108 (shown in a pump housing) and a flow sensor 110 (shown a housing), are mounted in relation to one another. The substrate 118 includes channels 122a, 122b, 122c, and 122d that connect each of the components of the microfluidic chip 100 to form a fluid loop. The fluid loop can also include ports 120a-c for sampling fluid media from the fluid loop. The pump 108 is configured to recirculate fluid media through the channel 122a through the cell chamber 104, through the channel 122d, through the flow rate sensor 110, though the channel 122b to the reoxygenation chamber 106, through the channel 122c and back to the pump 108. The fluid loop includes oxygen sensors 124a and 124b near the input port 112a and the output port 112b, respectively, the of the cell chamber 104.

The microfluidic chip 100 includes a removable cell chamber insert 102 that houses the cell chamber 104. The cell chamber insert 102 is removable from the microfluidic chip 100. The removable cell chamber insert 102 enables different types of cell chambers or instances of liver tissues to be included in the fluid loop of the microfluidic chip without requiring replacement of all other components.

The cell chamber insert 102 can be fastened to the microfluidic chip 100 using fasteners. The cell chamber insert 102 is removable to enable a user to seed and/or perfuse cells and tissue in the cell chamber 2024 away from the microfluidic chip 100. The liver tissue can be seeded and perfused in a sealed environment in the cell chamber 104. Once the tissue is prepared, the cell chamber insert 102 can be added to the microfluidic chip 100. In some implementations, the microfluidic chip 100 is reusable with different instances of the insert 102, while the insert itself is disposable. Different examples of the insert 102 are described in further detail herein. For example, the insert 102 can be a top-mounted insert mounted on top of substrate 118 or a bottom-mounted insert mounted onto a bottom layer of the microfluidic chip 100. Examples of these inserts can be found in WO Pub. No. 2023/023652, filed on Aug. 19, 2022, the entire contents of which are incorporated by reference herein.

The cell chamber 104 hosts the tissue for emulation of tissue functionality. For example, the tissue is configured to experience an emulated in vitro environment within the cell chamber 104. The cell chamber 104 portion can include a semi-permeable membrane that hosts a cell/tissue culture. The insert enables the tissue to be cultured independently from the microfluidic chip 100, and then added to the fluid loop once the tissue is mature for experimentation. For example, a first cell culture can be swapped for a second cell culture without replacing the remaining hardware (e.g., the pump 108, flow sensor 110, etc.) on the microfluidic chip 100. Fluid enters the cell chamber 104 at a port 112a near oxygen sensor 124a and exits at port 112b near an oxygen sensor 124b.

The microfluidic chip 100 is configured to measure the oxygen concentration of the fluid media prior to entering the cell chamber 104 and after the fluid media exits the cell chamber 104. The difference between the oxygen concentration levels measured at sensors 124a and 124b can help describe any damage that has occurred to the tissue within the cell chamber 104 that may be caused by the drug in the fluid media.

The microfluidic chip 100 includes a pump 108 configured to circulate the fluid media through the fluid loop of the microfluidic chip. The pump 108 receives fluid through an inlet port and pumps the fluid through an outlet port through a fluid channel 122a. The pump 108 receives fluid from the reoxygenation chamber 106. The pump 108 is coupled to the substrate 118 of the microfluidic chip 100 by a housing. For example, an unexpected drop (or lack of a drop) in the oxygen concentration level can signify that the tissue has been damaged by the drug within the fluid media. The relationship between oxygen concentrations and cell damage is subsequently described in further detail.

A reoxygenation chamber insert 116 hosts the reoxygenation chamber 106. The reoxygenation chamber insert 116 can be removed and replaced on the microfluidic chip 100, similar to the cell chamber insert 102. The reoxygenation chamber 106 is configured to enable oxygen to mix with the fluid to reintroduce oxygen at specified concentrations to the fluid media of the fluid loop. A port 130 enables pressure equilibration for the microfluidic chip 100, while a permeable membrane of the chamber 106 enables air to enter the chamber and mix with the fluid media, dissolving in the fluid media and reoxygenating the fluid media (as subsequently described). The reoxygenation chamber 106 is connected to a sampling port (e.g., port 120c) that enables samples of the fluid media to be removed from the fluid loop for analysis. For example, the oxygen concentration, drug concentration, etc. of the fluid media can be measured by extracted fluid through the sampling port 120c. This can enable analysis of how much oxygen has been absorbed by the tissue in the cell chamber 104 or how much of a drug has been absorbed by the cells of the cell chamber 104.

The reoxygenation chamber 106 is configured to add oxygen to the fluid media in a precise concentration. The concentration emulates a concentration of oxygen that would be available to liver tissue in the cell chamber 104 if the tissue were in the human body. The pump 108 pumps the fluid media through the fluid loop at a controlled rate (e.g., 1-2 mL/hour). The flow rate is set to a slow flow rate to minimize shear experienced by the tissue in the cell chamber 104. The fluid media is pumped from the pump 108 through the cell chamber 104, through the flow sensor 110, through the reoxygenation chamber 106, and back to the pump 108. As previously described, the fluid recirculates though the cell chamber 104 repeatedly in a closed, recirculating fluid loop.

The reoxygenation chamber 106 is configured to provide a particular concentration of oxygen to the fluid media. The reoxygenation chamber 106 can be modular with respect to the rest of the microfluidic chip 100 via insert 116. For example, the reoxygenation chamber 106 can be fastened to the substrate 118 with fasteners. The reoxygenation chamber 106 can be removed and replaced with another instance of the reoxygenation chamber without replacing the other hardware elements (e.g., the pump 108, flow sensor 110, etc.) of the microfluidic chip 100.

The microfluidic chip 100 includes several ports for adding or removing cells, drugs, or fluid media to the fluid loop. The microfluidic chip 100 includes a fill port that enables a user to fill the fluid loop with fluid media. The microfluidic chip 100 includes seeding ports. In some implementations, a first seeding port enables a user to seed an apical side of the cell chamber 104, and the seeding port enables a user to seed a basal side of the cell chamber 104. The tissue of the cell chamber 104 can be seeded directly within the cell chamber 104, rather than being cultured in another environment and later added to the cell chamber. Generally, the components of the ports 120a-c otherwise include molded thermoplastic. A vent (not shown) can be introduced to enable air to escape from the fluid loop as fluid media is introduced.

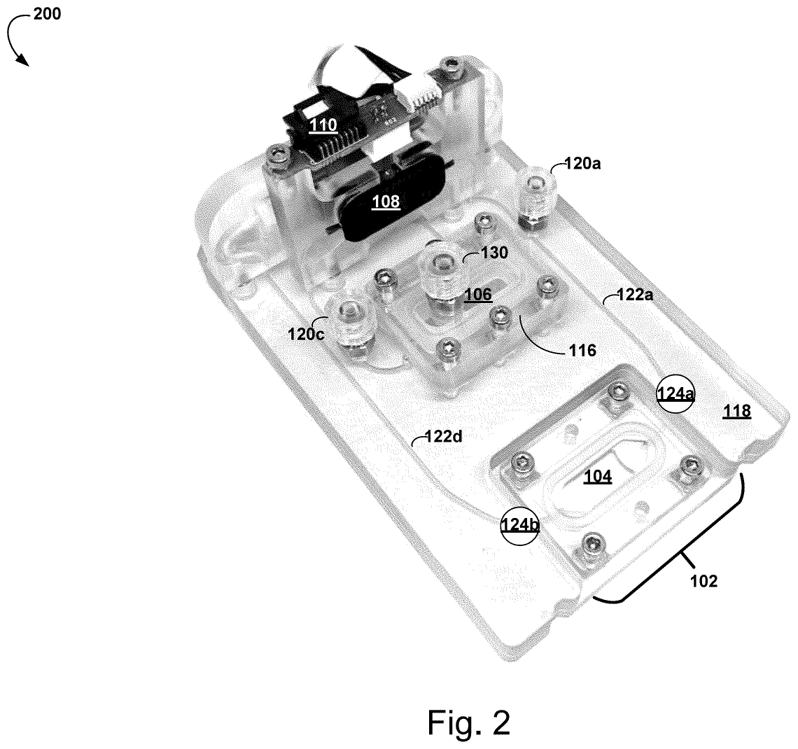

FIG. 2 shows a perspective view of an example microfluidic chip 200 for performing drug assays on tissue to enable generation of estimates for liver-specific DILI parameters. The microfluidic chip can be the similar to microfluidic chip 200 of FIG. 1. The microfluidic chip 200 can be formed from a plurality of layers that are mechanically fastened together. In some implementations, the layers, including substrate 118, are formed from thermoplastic. In some implementations, the thermoplastic includes cyclic olefin copolymer (COC)-based thermoplastic. In some implementations, the thermoplastic includes polycarbonate (PC)-based thermoplastic. At least two layers of the thermoplastic material are heat bonded together, forming the fluid passageways 122a-d and chambers 104, 106 of the recirculation loop.

Construction from the thermoplastic, as opposed to a polydimethylsiloxane (PDMS)-based plastic, provides several advantages. The thermoplastic material enables the microfluidic chip 200 to be formed from layers of thermoplastic that are mechanically coupled together (e.g. with screws). Construction of the entire fluid loop from the thermoplastic, as opposed to only a portion of the fluid loop such as the cell chamber 104, enables recirculation of fluid through a fluid loop that includes the hosted tissue culture. Recirculation of the fluid through the fluid loop enables high fidelity emulation of human physiology. Fluid can be pumped (e.g., by pump 108) through or across the tissue to the reoxygenation chamber 106 that adds a precise amount of oxygen to the fluid media without any exposure to oxygen outside of the fluid loop. The fluid media is then re-pumped through the cell chamber 104. The closed loop configuration enables more accurate testing of drug absorption in the tissue in comparison with open loop configurations in which fluid media is pumped across a cell chamber once. The closed fluid loop enables extended emulation of human physiology for a tissue sample. The closed fluid loop can be automatically sampled at various points in time over a course of hours, days, weeks, months, and so forth.

The thermoplastic construction of the fluid loop enables recirculation of the fluid in a closed fluid loop without drug adsorption into the thermoplastic. Adsorption includes an adhesion of the drug (e.g., a lipophilic drug) or fluid media to the channels of the microfluidic chip 200. This process creates a film of the drug on the surface of the fluid channels and within the cell chamber 104. For example, PDMS can absorb up to 100% of drugs in the fluid loop if the microfluidic chip 200 is constructed from PDMS when the fluid is pumped quickly through the cell chamber. The thermoplastic material of the microfluidic chip 200 enables testing of a wider variety of drugs with higher precision relative to PDMS-based microfluidic chips. Because drug adsorption is limited, the thermoplastic material of the microfluidic chip 200 enables a fast flow fluid loop (e.g., several mL/hour), mimicking a human physiology and enabling accurate emulation of drug absorption in the human body. For example, the cell chamber geometry enables a reduced shear on the cells of the tissue in the fluid loop (even for fast flow recirculating systems), relative to a higher shear experienced by cells in other cell chamber geometries. A lower shear improves testing outcomes and destroys fewer cells in the tissue. Flow rates for PDMS-based cell chambers are relatively slower to reduce shear on the cells. The microfluidic chip 200 enables higher oxygenation rates relative to PDMS-based flow through systems that do not have a waterfall feature. This raises oxygenation levels supplied to the cells in the cell chamber relative to those PDMS-based flow through systems and avoids cell death near the cell chamber exit (e.g., caused by depleted oxygen levels near the cell chamber exit).

The microfluidic chip 200 is configured for modeling tissue damage due to drug absorption by enabling emulation of liver tissues in controlled environments. To perform a test, there can be an induction of damage in the cells of the microfluidic chip 200 in the prepared liver tissues, such as due to a drug in the fluid media. The damage progression can be monitored over long periods of time.

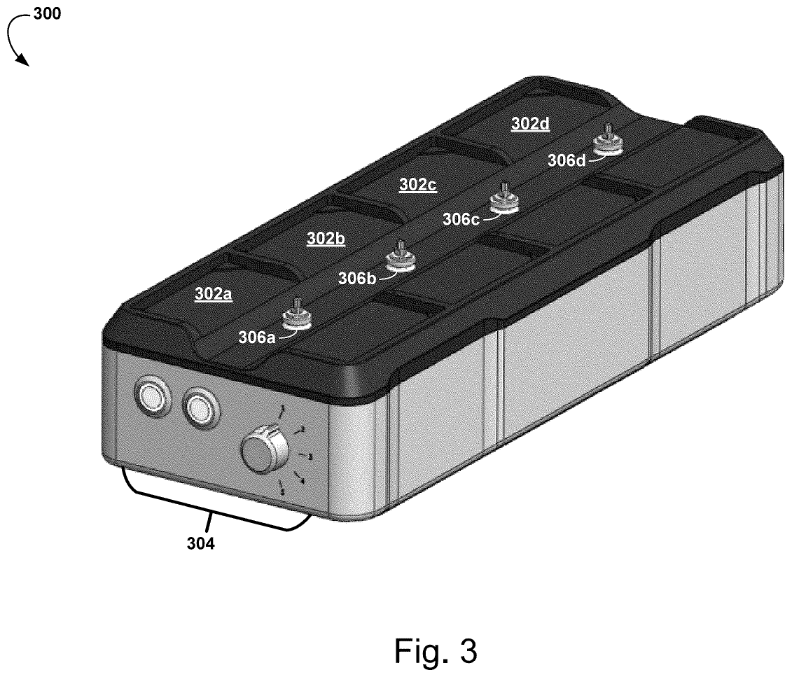

FIG. 3 shows a perspective view of an example platform 300 configured to host a plurality of microfluidic chips shown in FIGS. 1-2 for performing drug assays on tissue to enable generation of estimates for liver-specific DILI parameters. The platform 300 can be configured to connect to a plurality of instances of the microfluidic chip 100. In the example of the platform 300, up to four microfluidic chips can be hosted in corresponding bays 302a, 302b, 302c, and 302d. In some implementations, each of the microfluidic chips 100 can include a sampling carousel (not shown in FIGS. 1-2) configured to take samples of the fluid media from the fluid loop of each microfluidic chip. In this example, the sampling carousel of the microfluidic chip 100 can be advanced using the respective motors 306a-d in the platform to take a plurality of samples automatically. Examples of sampling carousel can be found in WO Pub. No. 2023/023652, filed on Aug. 19, 2022, the entire contents of which are incorporated by reference herein.

A user interface 304 of the platform 300 enables a user to cause automated drug assays of the tissues hosted in respective microfluidic chips on the platform 300. The user interface 304 can include a dial, buttons, a touchscreen, or other input devices that enable the user to select a particular microfluidic chip for controlling recirculation of fluid media, sampling, or one or more other functions of the assay. In some implementations, the platform is configured to remotely communicate with a data processing system in which the user can wirelessly transmit instructions to the platform 300 by a user interface on a remote computing device. The platform 300 enables a user to conduct high throughput assay analysis of multiple instances of the microfluidic chip in an automated fashion. While four bays 302a-d are shown, the number of bays can vary such as from two to ten bays. In some implementations, one or more controllers associated with the platform enable a simplified end-user interface. The platform 300 can be a plug-and-play platform and can include of a simplified chip and controller interface with ribbon connector, an independent flow-controller for each chip, a touch screen to enter experimental parameters, and continuous performance recording to a memory card.

The microfluidic chip 100 can include a liver tissue chip to assess multiple DILI mechanisms in a single experiment using clinically relevant metrics. The microfluidic chip 100 recapitulates human tissue functions in vitro and eliminates species-specific differences since the microfluidic chip 100 uses primary human hepatocytes and non-parenchymal cells. The microfluidic chip 100 enables testing with micrograms of a drug without requiring an API scale-up, as previously described. The tissues are metabolically active and can metabolize drugs at a similar rate to what is observed in humans, as subsequently described. The microfluidic chip 100 with liver tissue supports an extended culture duration of up to 28 days, enabling the proposed studies which use 3-5 days of tissue maturation and 15 days of drug testing. In contrast to systems using spheroid cultures, the microfluidic chip 100 enables time for the parent drug to be cleared and toxic metabolites to form. In addition, multiple readouts can be obtained over time, including levels of the drug and metabolites, clinical metrics (ALT, AST, ALP, albumin, urea, and bile acids), tissue analysis, and functional studies, both through the platform 300 or directly from the microfluidic chip 100. Thus, the microfluidic chip 100 with liver tissue enables simultaneous data for multiple mechanisms to be obtained to inform DILI liability decision-making. The microfluidic chip 100 enables efficiency to drug development productivity by improving the quality of DILI assessment, accelerating discovery timelines, and reducing the cost.

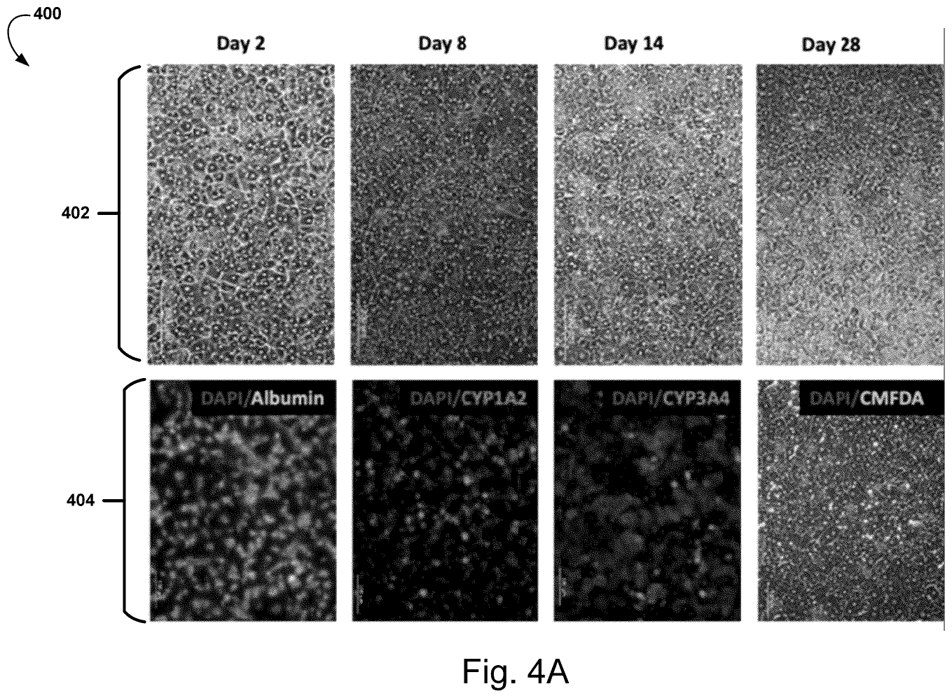

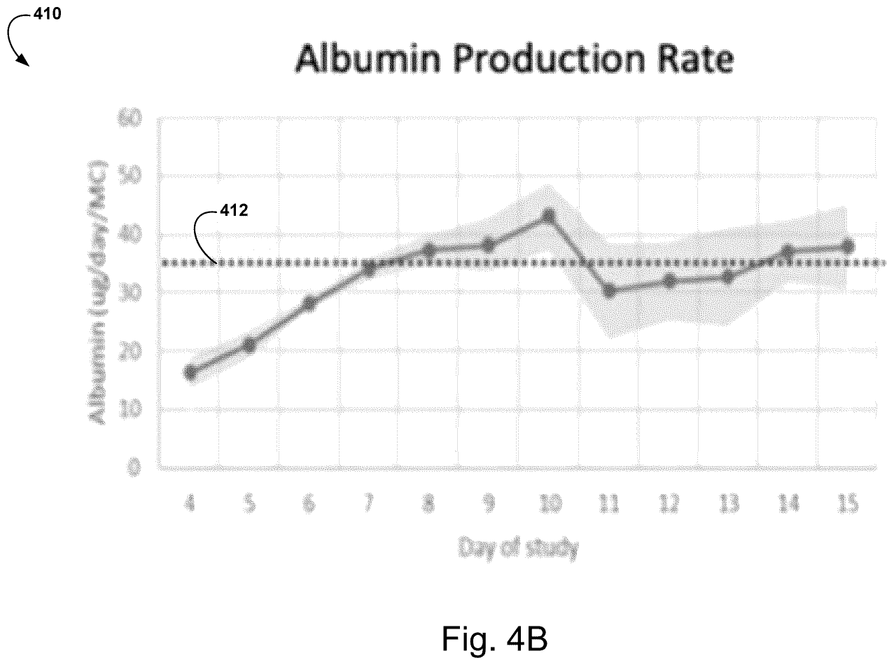

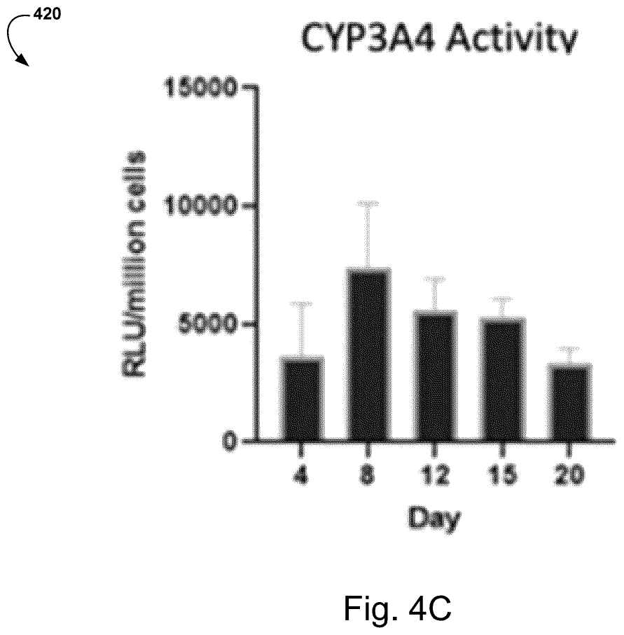

The microfluidic chip 100 enables milli-fluidic tissue chip perfusion that maintains mature liver tissue with functional enzymes and transporters and allows reliable, reproducible, validated, and cost-effective prediction of DILI. The microfluidic chip 100 metabolize drugs similar to clinical metabolism rates and that they are more functional and informative than rat studies and cellular hepatocyte cultures, as described in relation to FIGS. 4A-4C. The microfluidic chip 100 with liver tissue for DILI assessment integrates the oxygen sensors 124a-b for assessment of mitochondrial function as a DILI mechanism and then enables performance of a dose-escalation validation study using a panel of 20 drugs with different hepatic clearance mechanisms and DILI mechanisms and severity.

FIGS. 4A, 4B, and 4C show results data that illustrate a functionality of the microfluidic chip 100 of FIGS. 1-2. In this example, a liver tissue microfluidic chip forms physiologically relevant liver tissue by 7 days and the cells can be viable for at least 28 days. The data show sustained hepatic tissue morphology and function in the microfluidic chip 100. To assess hepatic clearance by the microfluidic chip 100, the microfluidic chip achieves accurate liver morphology and functionality characteristics. By seeding primary hepatocytes as a sandwich culture (˜215,000 cells per chamber), the microfluidic chip maintains hepatocyte morphology for at least 28 days, with a cell seeding yield of over 90%.

Images of FIG. 4A show cell viability over time. Images 402 show that primary human hepatocytes (PHH) cells were seeded in a sandwich culture and grown in an instance of the microfluidic chip 100. Immunohistochemistry (IHC) indicates that a presence of albumin, the cytochrome P450 enzymes CYP1A2 and CYP3A4, and bile canaliculi (CMFDA) are present for at least 28 days. Images 404 show IHC staining for the liver-specific markers albumin, CYP1A2, CYP3A4, and CMFDA after 7 days of culture. DAPI (4′,6-diamidino-2-phenylindole) was used to counter-stain nuclei. The liver-specific proteins (albumin, the cytochrome P450 [CYP] enzymes CYP2A1 and CYP3A4, and bile canaliculi [CMFDA]) were observed by IHC at day 28, confirming that the tissue is physiologically relevant. The cell cultures allow comprehensive testing of both fast and slow cleared drugs, as well as multiple hepatic clearance mechanisms. To confirm these observations, the microfluidic chip assessed the rate of albumin production over time using ELISA for albumin, as shown in graph 410 of FIG. 4B.

FIG. 4B shows a graph 410 representing albumin levels associate with the microfluidic chip over time. Albumin levels were assessed in the microfluidic liver chip system daily on days 4-15 by an enzyme-linked immunosorbent assay (ELISA). Clinical production rates are shown using the dashed line 412. Samples can be measured daily on days 4-15 of culture. In this example, the production of albumin is maintained at clinical production rates until at least day 15.

FIG. 4C shows a graph 420 representing cytochrome P450 CYP3A4 activity. The activity was assessed by luminescent assay and showed sustained enzymatic activity over three weeks of culture. The microfluidic chip system confirms a presence of CYP metabolic activity in instances of the microfluidic chip 100. Activity is maintained for at least 20 days in this example. The microfluidic chip 100 produces functional tissue with albumin production rates as observed in humans, with sustained CYP activity.

There are minimal non-specific binding and evaporative losses. To assess non-specific binding, chips without liver tissue were incubated with a variety of neutral, basic, and acidic drugs for 24 hours and the drug concentration in the system was measured using LC/MS. Unlike control PDMS chips, the microfluidic chip, based on COC construction, exhibited minimal non-specific binding.

As previously described, the microfluidic chip 100 includes oxygen sensors 124a-b for real-time evaluation of mitotoxicity. The oxygen sensors 124a-b can enable in vitro assessment of DILI. The microfluidic chip 100 and control system perform measurements of the oxygen consumption rate of in vitro liver tissue as a readout of mitochondrial function.

Oxygen sensing is achieved by combining a disposable oxygen sensor spot with an oxygen sensing meter. The meter provides an optical excitation signal to the sensor spot and measures the emitted light from the sensor spot. The light emitted by the sensor spot is influenced by a partial pressure of oxygen in fluid in contact with the sensor spot. Two sensor spots are placed in the primary flow path either side of the tissue culture area to measure the oxygen consumption of the tissue. The meters in the control module are placed such that they can measure the oxygen sensor values during normal operation. Oxygen meters can be read by a data processing system using wireless or wired messages, stored/read from a database, and so forth associated with the platform 300.

The microfluidic chip 100 performs quantification of cellular respiration to assess mitochondrial function. One of the last steps of cellular respiration is the oxidation of cytochrome c in complex IV, which reduces oxygen to form water. Estimations of OCR are conclusive for the ability to synthesize ATP and mitochondrial function even more than measurements of intermediates (such as ATP or NADH) and potentials. To assess the sensitivity of the oxygen sensors 124a-b and on-chip OCR estimates, the microfluidic chip 100 performs a mitochondrial stress test. After cells are seeded on chips (e.g., 4 per treatment), tissue is matured (˜5-7 days), and basal OCR is established, oligomycin (1 μM & 30 min), which blocks proton translocation through complex V and represses ATP and OCR, are administered to the chips and OCR are assessed continuously. The microfluidic chip enables the tissue to be treated with FCCP (0.3 μM & 30 min), which dissipates the gradient uncoupling electron transport from complex V activity and increases OCR to a maximum level. Mitochondrial respiration can be completely inhibited using rotenone (1 μM) and antimycin A (0.5 μM) for 30 min to inhibit complex 1. The oxygen sensors 124a-b read oxygen levels on the fluid path before and after the cell chamber. The inlet readout represents oxygenated media levels leaving the reoxygenation channel and the outlet readout reflects oxygen-depleted media due oxygen consumption of liver tissue. The continuous readouts are captured and recorded by the controller 114 of the microfluidic chip 100.

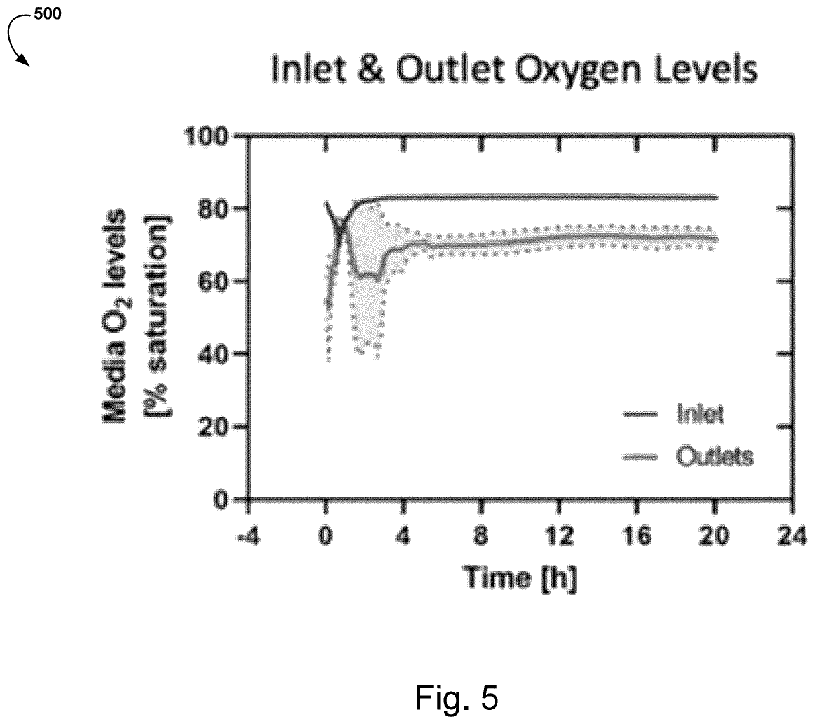

FIG. 5 shows a graph 500 of example oxygen levels with the microfluidic chip of FIGS. 1-2. The media oxygen levels can be sampled periodically or continuously over time, both intraday and over the course of several weeks.

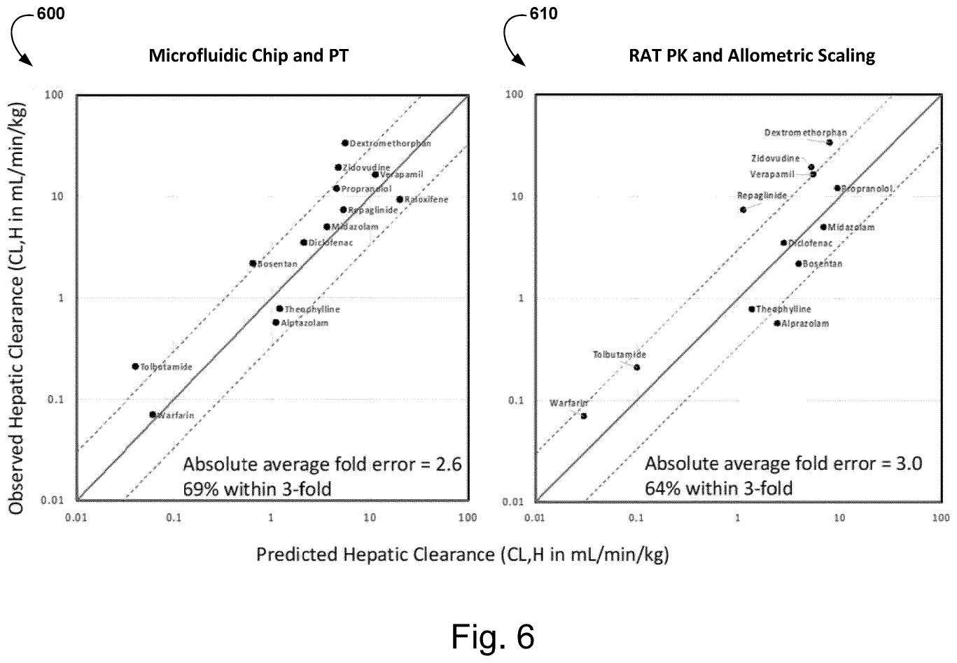

FIG. 6 shows graphs 600, 610 representing examples predicted and observed hepatic clearances. Generally, there is a high correlation of hepatic clearance to clinical observations. In vitro CLint,u values are converted to CIh (hepatic clearance scaled to human size) using a parallel tube model. Graph 600 shows data obtained using the microfluidic chip 100. Graph 610 shows examples of published rat PK studies [Wood, Houston, Hallifax 2017]. Solid lines are the unity line (perfect prediction) and dashed red lines are 3-fold error. Overall, the microfluidic chip 100 predicts clinical clearance better than most common in vivo studies. (AAFE=2.6 vs 3.0. Within 3-fold error 69 vs 64%).

As shown in graph 600, oligomycin treatment results in decreased OCR values, but non-zero values since non-mitochondrial respiration is independent of this treatment. FCCP treatment increases OCR to its maximum level, and a higher oxygen gradient on the sensors on the microfluidic chip can be observed. Rotenone treatment leads to complete reduction in OCR, and similar oxygen levels can be observed between the sensors. As an alternative, the second (outlet) sensor location on the cell chamber can be optimized to further improve its sensitivity to detect oxygen.

The microfluidic chip is configured for an assessment of hepatoxic responses MPS IQ consortium DILI drug list. A co-culture of primary human hepatocytes, Kupffer cells, and stellate cells are cultured in the microfluidic chip 100, incubated with four doses of each drug for four days, and assessed for a variety of physiologic parameters including clinical metrics (ALT, AST), tissue health parameters (urea, albumin), and functional studies (oxygen consumption, and cell viability). The total drug treatment can be 16 days, beginning on day 5, for a total experimental period of 21 days. All these parameters can be assessed on a single instance of the microfluidic chip 100.

For assessing DILI, the tissue is confirmed to be metabolically active. Sustained CYP activity was demonstrated over at least 3 weeks of culture in the microfluidic chip 100, indicating that the tissues are metabolically functional. In addition, a set of drugs can be tested across a range of high to low clearance that are substrates to various metabolizing enzymes to assess in vitro drug clearance rates. The in vitro results can be translated to a human scale based on the relative tissue sizes and compared to clinically-observed hepatic clearance rates (CLh). The results of graph 600 and FIGS. 4A-4D demonstrate that the microfluidic chip 100 metabolizes drugs at similar rates as observed in humans. These results are compared to the literature values from rat studies as shown in FIG. 6. The microfluidic chip 100 improves prediction power relative to existing studies.

PHH is cultured to form a confluent monolayer, then Kupffer cells (KC) and hepatic stellate cells (HSC; Novabiosis & BioIVT) are introduced in a Matrigel overlay enabling close cell-cell interactions replicating human liver cytoarchitecture. 215,000 PHH, 47,000 KC, and 18,000 HSC are seeded per chip resulting in an approximate ratio of 12:3:1 (PHH:KC:SC), closely resembling human liver cell composition.

Example drugs for testing are shown in Table 1. These drugs are selected based on recommendations made by the IQ Consortium, a pharmaceutical and biotechnology association that aims to advance innovation and quality in the biopharmaceutical industry. However, any drug can be tested using the microfluidic chip 100 (e.g., hundreds or thousands of different drugs). This list is intended as an illustrative example and not an exhaustive set.

TABLE 1
Example toxic and less toxic drugs for testing toxic

| Liver | DILI | Mechanism | Less toxic | DILI | Comparator |
|---|---|---|---|---|---|
| toxicant | presentation | of toxicity | comparator | presentation | characteristic |
| Sitaxsentan | ALT elevations | Reactive | Ambrisentan | Minimal | Targets the |
| | after 2 weeks | metabolites, | | ALT | same receptor |
| | | mitochondrial | | elevations | as sitaxsentan |
| | | toxicity, | | | |
| | | BSEP | | | |
| | | inhibition | | | |
| Clozapine | ALT elevations | Reactive | Olanzapine | No DILI | Structurally |
| | after 1 week | metabolite | | concerns | similar to |
| | | | | | clozapine |
| Diclofenac | ALT elevations | Reactive | | | |
| | within 1 month | metabolites, | | | |
| | | mitochondrial | | | |
| | | dysfunction, | | | |
| | | bile acid | | | |
| | | dysfunction | | | |
| Zileuton | ALT elevations | Reactive | | | |
| | after 6 weeks | metabolite | | | |
| | | formation | | | |
| Fialuridine | Liver failure | Mitochondrial | FIRU | No DILI | Stereoisomer |
| | after 12 weeks | toxicity as | | concerns | of fialuridine |
| | of dosing | primary event | | | |
| | | causing lactic | | | |
| | | acidosis, | | | |
| | | microvesicular | | | |
| | | steatosis | | | |
| Tolcapone | ALT elevations; | Reactive | Entacapone | Low DILI | Similar BSEP |
| | acute liver | metabolite, | | concern | profile, but |
| | failure | mitochondrial | | | less |
| | | toxicant, | | | mitochondrial |
| | | BSEP | | | toxicity |
| | | inhibition | | | |
| Asunaprevir | ALT elevations | Alterations in | | | |
| | after 2 weeks | bile acids | | | |
| Troglitazone | ALT, bilirubin | Reactive | Pioglitazone | Low DILI | Low clinical |
| | elevations after | metabolites, | | concern | dose |
| | 18 weeks | BSEP | | | |
| | | inhibition | | | |
| Telithromycin | ALT elevations | Bile acid | | | |
| | after 1 day | alterations | | | |
| Trovafloxacin | Acute liver | Immune- | Levofloxacin | No/low | Same |
| | failure | mediated | | DILI | structural |
| | | | | concern | class as |
| | | | | | trovafloxacin |
| Pemoline | Liver failure | Immune- | | | |
| | | mediated | | | |
| Mipomersen | Oligonucleotide, | Lipid | | | |
| | ALT elevations | alterations | | | |
| | and hepatic | | | | |
| | steatosis | | | | |
| Nefazodone | Liver failure | Reactive | Buspirone | No/low | Less BSEP |
| | | metabolites, | | DILI | inhibition and |
| | | BSEP | | concern | weak-no |
| | | inhibitor, | | | mitochondrial |
| | | mitochondrial | | | toxicity |
| | | tox | | | |

The microfluidic chip 100 can test drugs using a dose escalation process. In an example, the microfluidic chip 100 hosting the liver tissue is seeded and matured for 5 days with daily partial media changes (25% of total media). Samples (˜400 μL per timepoint) are stored for the assays. At day 5, the first dose (1×Cmax) is added with complete media change (1.8 mL media) and incubate the drug for 4 days without any new media addition. Daily samples of 25 L are collected for LDH assessment using a colorimetric assay. At the end of each 4-day drug treatment, all the media (>1.5 mL) are collected and quantified for albumin, ALT, AST, ALP (using standard ELISAs from AbCam), urea (Quantichrom™ urea assay kit; Bioassay Systems), and bile acids (fluorometric kit; Sigma-Aldrich). Before adding the next dose, a non-invasive viability assay (PrestoBlue) is run for 1 hour; then, add the new dose for the next 4 days. The same assays are repeated on days 5 (pre-dose), 9, 13, 17, and 21. At day 21, live/dead staining is performed as the endpoint assay (LIVE/DEAD™; ThermoFisher).

The doses are based on clinically observed maximum drug concentrations (Cmax) in human plasma. Using clinical information, the data processing system determines an unbound (effective) drug concentration in a human and the in vitro dose. For example, the Diclofenac Cmax is 4-10 μM, but it is 99% protein-bound (only 1% unbound). Experiments can be performed in batches such that each batch has one untreated control and 5-7 drugs and is run for 21 days. Structurally similar drugs can be included with high and less DILI concerns (Table 1), such as tolcapone and entacapone, respectively, in the same batch. Each study can have four chip replicates.

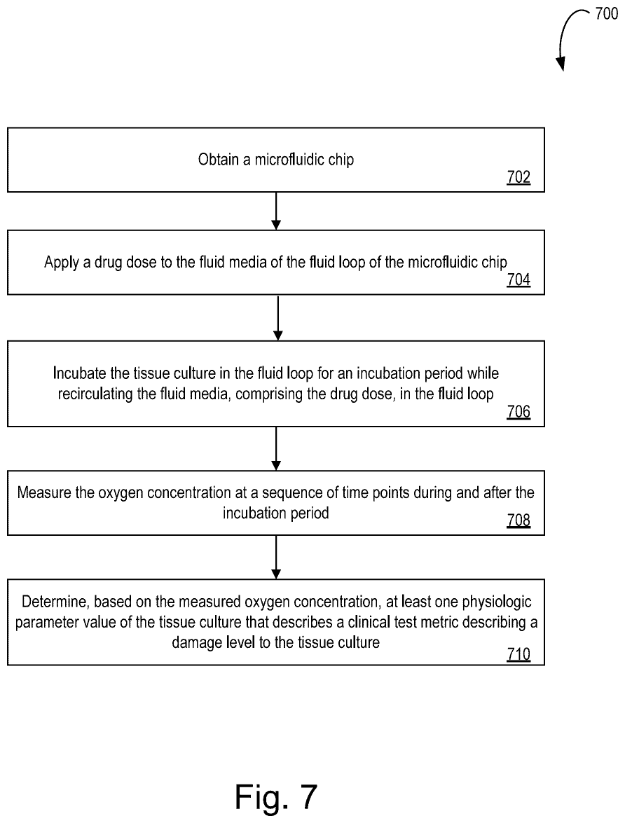

FIG. 7 is a flow diagram illustrating example process 700 for performing drug assays on tissue to enable generation of estimates for liver-specific DILI parameters. The process 700 can be performed using the microfluidic chips and systems described herein. The process 700 includes obtaining (702) a microfluidic chip. The microfluidic chip can be the microfluidic chip 100 of FIG. 1 and includes a cell chamber hosting a tissue culture comprising liver tissue. The microfluidic chip includes a reoxygenation chamber configured to add oxygen to a fluid media. The microfluidic chip includes a fluid loop configured to recirculate the fluid media through the cell chamber and the reoxygenation chamber and supply oxygenated fluid media to the tissue culture. The microfluidic chip includes at least one oxygen sensor configured to measure an oxygen concentration within the fluid media. The process 700 includes applying (704) a drug dose to the fluid media of the fluid loop of the microfluidic chip. The process 700 includes incubating (706) the tissue culture in the fluid loop for an incubation period while recirculating the fluid media, comprising the drug dose, in the fluid loop. The process 700 includes measuring (708) the oxygen concentration at a sequence of time points during and after the incubation period. The process 700 includes determining (710), based on the measured oxygen concentration, at least one physiologic parameter value of the tissue culture that describes a clinical test metric describing a damage level to the tissue culture.

In some implementations, the clinical test metric includes one or more of alanine transaminase (ALT), aspartate transaminase (AST), alkaline phosphatase (ALP), gamma-glutamyl transferase (GGT), serum bilirubin, prothrombin time (PT), an international normalized ratio (INR), a total protein, and an albumin.

In some implementations, the at least one physiologic parameter value of the tissue culture represents a cell viability metric value for the tissue culture.

In some implementations, a media volume in the microfluidic chip includes at least 2 milliliters (ml).

In some implementations, the microfluidic chip is configured for intracellular and extracellular pharmacological characterization on a single microfluidic chip.

In some implementations, the fluid loop comprises a cyclic olefin copolymer (COC) that is configured to reduce or eliminate a non-specific binding of lipophilic drugs.

In some implementations, the microfluidic chip is translucent or transparent, and wherein the microfluidic chip does not cause auto-fluorescence responsive to fluorescence imaging in the cell chamber.

In some implementations, the cell chamber includes a tissue surface area of at least 1 square centimeter (cm2).

In some implementations, the at least one oxygen sensor comprises a first oxygen sensor configured to measure the oxygen concentration in the fluid media at an inlet of the cell chamber, and wherein the at least one oxygen sensor comprises a second oxygen sensor configured to measure the oxygen concentration in the fluid media at an outlet of the cell chamber.

In some implementations, the process includes maintaining the tissue culture for at least 21 days.

In some implementations, the tissue culture comprises at least 200,000 cells.

In some implementations, the process includes determining a mitochondrial function based on the measured oxygen concentration of the tissue culture.

In some implementations, the drug dose comprises one of Sitaxsentan, Clozapine, Diclofenac, Zileuton, Fialuridine, Tolcapone, Asunaprevir, Troglitazone, Telithromycin, Trovafloxacin, Pemoline, Mipomersen, or Nefazodone.

In some implementations, the process includes applying a series of drug doses to the fluid media of the fluid loop of the microfluidic chip during and after the incubation period, the series of drug doses increasing in value.

In some implementations, the process includes applying a series of drug doses to the fluid media of the fluid loop of the microfluidic chip every four days.

In some implementations, the process includes obtaining a plurality of instances of the microfluidic chip; applying drug doses to each microfluidic chip instance of the plurality, a type of the drug dose being different for each microfluidic chip instance; and controlling the plurality of instances of the microfluidic chip in parallel.

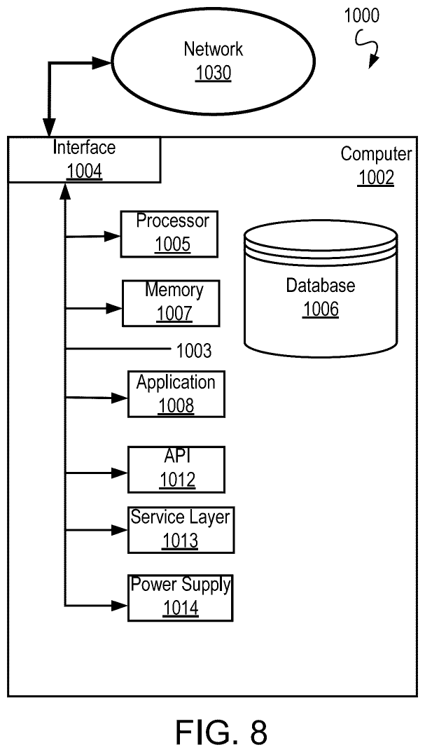

FIG. 8 is a block diagram of an example computer system 1000 used to provide computational functionalities associated with described algorithms, methods, functions, processes, flows, and procedures described in the present disclosure (such as the method 700 described previously with reference to FIG. 7), according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI or GUI).

The computer 1002 can serve in a role as a client, a network component, a server, a database, a persistency, or components of a computer system for performing the subject matter described in the present disclosure. The illustrated computer 1002 is communicably coupled with a network 1030. In some implementations, one or more components of the computer 1002 can be configured to operate within different environments, including cloud-computing-based environments, local environments, global environments, and combinations of environments.

At a high level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1030 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1003. In some implementations, any or all of the components of the computer 1002, including hardware or software components, can interface with each other or the interface 1004 (or a combination of both), over the system bus 1003. Interfaces can use an application programming interface (API) 1012, a service layer 1013, or a combination of the API 1012 and service layer 1013. The API 1012 can include specifications for routines, data structures, and object classes. The API 1012 can be either computer-language independent or dependent. The API 1012 can refer to a complete interface, a single function, or a set of APIs.

The service layer 1013 can provide software services to the computer 1002 and other components (whether illustrated or not) that are communicably coupled to the computer 1002. The functionality of the computer 1002 can be accessible for all service consumers using this service layer. Software services, such as those provided by the service layer 1013, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1012 or the service layer 1013 can be stand-alone components in relation to other components of the computer 1002 and other components communicably coupled to the computer 1002. Moreover, any or all parts of the API 1012 or the service layer 1013 can be implemented as child or sub-modules of another software module, enterprise application, or hardware module without departing from the scope of the present disclosure.

The computer 1002 includes an interface 1004. Although illustrated as a single interface 1004 in FIG. 10, two or more interfaces 1004 can be used according to particular needs, desires, or particular implementations of the computer 1002 and the described functionality. The interface 1004 can be used by the computer 1002 for communicating with other systems that are connected to the network 1030 (whether illustrated or not) in a distributed environment. Generally, the interface 1004 can include, or be implemented using, logic encoded in software or hardware (or a combination of software and hardware) operable to communicate with the network 1030. More specifically, the interface 1004 can include software supporting one or more communication protocols associated with communications. As such, the network 1030 or the interface's hardware can be operable to communicate physical signals within and outside of the illustrated computer 1002.

The computer 1002 includes a processor 1005. Although illustrated as a single processor 1005 in FIG. 10, two or more processors 1005 can be used according to particular needs, desires, or particular implementations of the computer 1002 and the described functionality. Generally, the processor 1005 can execute instructions and can manipulate data to perform the operations of the computer 1002, including operations using algorithms, methods, functions, processes, flows, and procedures as described in the present disclosure.

The computer 1002 also includes a database 1006 that can hold data for the computer 1002 and other components connected to the network 1030 (whether illustrated or not). For example, database 1006 can be an in-memory, conventional, or a database storing data consistent with the present disclosure. In some implementations, database 1006 can be a combination of two or more different database types (for example, hybrid in-memory and conventional databases) according to particular needs, desires, or particular implementations of the computer 1002 and the described functionality. Although illustrated as a single database 1006 in FIG. 10, two or more databases (of the same, different, or combination of types) can be used according to particular needs, desires, or particular implementations of the computer 1002 and the described functionality. While database 1006 is illustrated as an internal component of the computer 1002, in alternative implementations, database 1006 can be external to the computer 1002.

The computer 1002 also includes a memory 1007 that can hold data for the computer 1002 or a combination of components connected to the network 1030 (whether illustrated or not). Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 10, two or more memories 1007 (of the same, different, or combination of types) can be used according to particular needs, desires, or particular implementations of the computer 1002 and the described functionality. While memory 1007 is illustrated as an internal component of the computer 1002, in alternative implementations, memory 1007 can be external to the computer 1002.

The application 1008 can be an algorithmic software engine providing functionality according to particular needs, desires, or particular implementations of the computer 1002 and the described functionality. For example, application 1008 can serve as one or more components, modules, or applications. Further, although illustrated as a single application 1008, the application 1008 can be implemented as multiple applications 1008 on the computer 1002. In addition, although illustrated as internal to the computer 1002, in alternative implementations, the application 1008 can be external to the computer 1002.

The computer 1002 can also include a power supply 1014. The power supply 1014 can include a rechargeable or non-rechargeable battery that can be configured to be either user- or non-user-replaceable. In some implementations, the power supply 1014 can include power-conversion and management circuits, including recharging, standby, and power management functionalities. In some implementations, the power-supply 1014 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1030. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

Particular implementations of the subject matter have been described. Other implementations, alterations, and permutations of the described implementations are within the scope of the following claims as are apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described.

Nevertheless, it are understood that various modifications may be made without departing from the spirit and scope of this disclosure.

What is claimed is:

1. A method for testing cell cultures for drug-induced liver injury (DILI), the method comprising:
obtaining a microfluidic chip comprising:
a cell chamber hosting a tissue culture comprising liver tissue;
a reoxygenation chamber configured to add oxygen to a fluid media;
a fluid loop configured to recirculate the fluid media through the cell chamber and the reoxygenation chamber and supply oxygenated fluid media to the tissue culture; and
at least one oxygen sensor configured to measure an oxygen concentration within the fluid media;
applying a drug dose to the fluid media of the fluid loop of the microfluidic chip;
incubating the tissue culture in the fluid loop for an incubation period while recirculating the fluid media, comprising the drug dose, in the fluid loop;
measuring the oxygen concentration at a sequence of time points during and after the incubation period; and
determining, based on the measured oxygen concentration, at least one physiologic parameter value of the tissue culture that describes a clinical test metric describing a damage level to the tissue culture.

2. The method of claim 1, wherein the clinical test metric includes one or more of alanine transaminase (ALT), aspartate transaminase (AST), alkaline phosphatase (ALP), gamma-glutamyl transferase (GGT), serum bilirubin, prothrombin time (PT), an international normalized ratio (INR), a total protein, and an albumin.

3. The method of claim 1, wherein the at least one physiologic parameter value of the tissue culture represents a cell viability metric value for the tissue culture.

4. The method of claim 1, wherein a media volume in the microfluidic chip includes at least 2 milliliters (ml).

5. The method of claim 1, wherein the microfluidic chip is configured for intracellular and extracellular pharmacological characterization on a single microfluidic chip.

6. The method of claim 1, wherein the fluid loop comprises a cyclic olefin copolymer (COC) that is configured to reduce or eliminate a non-specific binding of lipophilic drugs.

7. The method of claim 1, wherein the microfluidic chip is translucent or transparent, and wherein the microfluidic chip does not cause auto-fluorescence responsive to fluorescence imaging in the cell chamber.

8. The method of claim 1, wherein the cell chamber includes a tissue surface area of at least 1 square centimeter (cm²).

9. The method of claim 1, wherein the at least one oxygen sensor comprises a first oxygen sensor configured to measure the oxygen concentration in the fluid media at an inlet of the cell chamber, and wherein the at least one oxygen sensor comprises a second oxygen sensor configured to measure the oxygen concentration in the fluid media at an outlet of the cell chamber.

10. The method of claim 1, further comprising maintaining the tissue culture for at least 21 days.

11. The method of claim 1, wherein the tissue culture comprises at least 200,000 cells.

12. The method of claim 1, further comprising determining a mitochondrial function based on the measured oxygen concentration of the tissue culture.

13. The method of claim 1, wherein the drug dose comprises one of Sitaxsentan, Clozapine, Diclofenac, Zileuton, Fialuridine, Tolcapone, Asunaprevir, Troglitazone, Telithromycin, Trovafloxacin, Pemoline, Mipomersen, or Nefazodone.

14. The method of claim 1, further comprising applying a series of drug doses to the fluid media of the fluid loop of the microfluidic chip during and after the incubation period, the series of drug doses increasing in value.

15. The method of claim 14, further comprising applying a series of drug doses to the fluid media of the fluid loop of the microfluidic chip every four days.

16. The method of claim 1, further comprising:
obtaining a plurality of instances of the microfluidic chip;
applying drug doses to each microfluidic chip instance of the plurality, a type of the drug dose being different for each microfluidic chip instance; and
controlling the plurality of instances of the microfluidic chip in parallel.

17. A microfluidic chip comprising:
a cell chamber hosting a tissue culture comprising liver tissue;
a reoxygenation chamber configured to add oxygen to a fluid media;
a fluid loop configured to recirculate the fluid media through the cell chamber and the reoxygenation chamber and supply oxygenated fluid media to the tissue culture; and
at least one oxygen sensor configured to measure an oxygen concentration within the fluid media; and
a controller configured to perform operations comprising:
recirculating the fluid media in the fluid loop, the fluid media comprising a drug dose;
obtaining measurements of the oxygen concentration at a sequence of time points during recirculating; and
determining, based on the measured oxygen concentration, at least one physiologic parameter value of the tissue culture that describes a clinical test metric describing a damage level to the tissue culture.

18. The microfluidic chip of claim 17, wherein the clinical test metric includes one or more of alanine transaminase (ALT), aspartate transaminase (AST), alkaline phosphatase (ALP), gamma-glutamyl transferase (GGT), serum bilirubin, prothrombin time (PT), an international normalized ratio (INR), a total protein, and an albumin.

19. The microfluidic chip of claim 17, wherein the fluid loop comprises a cyclic olefin copolymer (COC) that is configured to reduce or eliminate a non-specific binding of lipophilic drugs.

20. The microfluidic chip of claim 17, wherein the at least one oxygen sensor comprises a first oxygen sensor configured to measure the oxygen concentration in the fluid media at an inlet of the cell chamber, and wherein the at least one oxygen sensor comprises a second oxygen sensor configured to measure the oxygen concentration in the fluid media at an outlet of the cell chamber.
